(19) United States
(12) Patent Application Publication
(10) Pub. No.: US 2024/0115791 A1
(43) Pub. Date: Apr. 11, 2024

(54) Suction Device With Improved Ejection Mechanism

(71) Applicant: Carilion Clinic, Roanoke, VA (US)

(72) Inventors: Robert W. Fippinger, Roanoke, VA (US); Trevor Owen, Roanoke, VA (US)

(21) Appl. No.: 18/482,398

(22) Filed: Oct. 6, 2023

Related U.S. Application Data
(60) Provisional application No. 63/414,108, filed on Oct. 7, 2022.

(51) Int. Cl. A61M 1/00

(52) U.S. Cl. CPC A61M 1/74; A61M 1/76

(57) ABSTRACT

A suction device is provided that includes a clearance mechanism that includes features that may facilitate its use by the surgeon with a single hand, thereby freeing up the other hand during a surgical operation.

### BACKGROUND

Surgeons often need to withdraw biological fluids from a surgical site during the course of surgery. Removing such materials has been typically conducted using a surgical suction device. One commonly used device for this purpose has been referred to as a Yankauer suction tubing.

One variation of surgical suction device includes an ejection wire that is designed to clear the tip of the suction device. U.S. Pat. No. 8,986,285 entitled Self-Cleaning Surgical Suction Device describes such an apparatus. A commercially available version of this device is shown in FIG. 1A has been referred to as the Chicago Tip. FIG. 1B from the shows a suction device having a suction tube 10, a guide structure 20, a bracket 30, a junction conduit 40, a tubular tip 50, a base member, and a handle member 100. A stylet (not shown) extends through the guide structure 20 and can be used to clear the tubular tip 50.

The present inventor has perceived a need for a surgical suction device that is capable of avoiding clogs from hard materials such as bone fragments and that can be used to conveniently dislodge such bone fragments during surgery.

### SUMMARY OF THE INVENTION

The present invention relates to a suction apparatus for the removal of debris from a surgical site. The suction device in one aspect of the invention includes (i) a suction tube having a suction opening at a distal end of the suction apparatus and having an interior defining a tubular suction channel for the removal of debris through suction at the suction opening, the suction tube bending toward a first direction; (ii) a handle portion adjacent the proximal end of the suction apparatus and connected to the suction tube; (iii) an ejection wire channel extending from a longitudinal opening in the suction apparatus to a wire exit opening that opens into the tubular suction channel of the suction tube; and (iv) a knob for controlling an ejection wire within the ejection wire channel, the knob being positioned on a side of the suction tube that is opposite the first direction in which the suction tube is bending.

In another aspect, the suction device is provided with various mechanisms for clog prevention that are used in connection with the ejection wire. In one case, the device may include a flow restriction. For example, the suction channel may include a third portion having a third cross-sectional area proximal to the second cross-sectional area, the third cross sectional area being larger than the second cross-sectional area. The suction device may use a gradual transition region between the first portion and the second portion, and the ejection wire channel may open in the gradual transition region. Alternatively, the suction channel may have a step transition between the first portion and the second portion, and the ejection wire channel opens in the first portion of the tubular suction channel.

In one aspect, the suction tip may include one or more lateral openings in the tubular suction channel proximate the opening at the distal end of the suction tip. The suction tip may also include lateral openings in the tubular suction channel at the distal end of the suction tip that define an offset ring structure. The suction device may include an ejection wire knob configured to slide back and forth within the longitudinal opening of the suction apparatus. The ejection wire knob may be configured to move the injection wire back and forth within the ejection wire channel.

In another aspect, the invention includes a suction apparatus for the removal of debris from a surgical site, the suction apparatus comprising: (i) a suction tube having a suction opening at a distal end of the suction apparatus and having an interior defining a tubular suction channel for the removal of debris through suction at the suction opening; (ii) a handle portion adjacent the proximal end of the suction apparatus and connected to the suction tube; (iii) an ejection wire channel extending from a longitudinal opening in the suction apparatus to a wire exit opening that opens into the tubular suction channel of the suction tube; and (iv) a knob for controlling an ejection wire within the ejection wire channel, wherein the knob has a range of travel that is 4 cm or less.

The present invention also includes methods of using the suction devices described herein during surgical procedures. These methods may include (a) removing at least biological fluids and bone fragments from the surgical site using the suction apparatus, and (b) ejecting one or more bone fragments collected in the first portion of the tubular suction channel using the ejection wire. For example, the additional biological fluids may be removed from the surgical site after (b) ejecting one or more bone fragment collected in the first portion of the tubular suction channel.

### BRIEF DESCRIPTION OF THE DRAWINGS

FIG. 1A shows a prior art suction device including a tip clearing mechanism.

FIG. 1B shows another prior art suction device including a tip clearing mechanism.

FIG. 2A shows a side view of a suction device according to an embodiment of the invention.

FIG. 2B shows cross section along axis A-A of FIG. 2A of the suction device according to an embodiment of the invention.

FIG. 2C shows a cut-out side-view of a suction device according to an embodiment of the invention.

FIG. 2D shows a cross section of the suction device shown in FIG. 2A according to an embodiment of the invention.

FIG. 2E shows a side view of the suction device according to an embodiment of the invention showing the slot where the knob is installed.

FIG. 2F shows a top-down view of the suction device according to an embodiment of the invention showing the slot where the knob is installed.

FIG. 2G shows a perspective view of the suction device according to an embodiment of the invention showing the slot where the knob is installed.

FIG. 3A shows a side view of the suction device according to another embodiment of the invention.

FIG. 3B shows a cut-out side view of the suction device of FIG. 3A.

FIG. 3C shows details around the tip of the suction device of FIG. 3A.

FIG. 3D shows a side view of the suction device of FIG. 3A.

FIG. 3E shows a cross-section E-E of the suction device of FIG. 3A.

FIG. 3F shows a cross-section F-F of the suction device of FIG. 3A.

FIG. 4A shows a perspective view of the knob details according to an embodiment of the invention.

FIG. 4B shows a view down the length of the knob of FIG. 4A.

FIG. 4C shows a side view of the knob of FIG. 4A.

FIG. 5A shows a view of an assembled suction device according to an embodiment of the invention in an extended state.

FIG. 5B shows a view of an assembled suction device according to an embodiment of the invention in a retracted state.

FIG. 5C shows a side view of the assembled suction device of FIG. 5A.

FIG. 5D shows a perspective view of the assembled suction device of FIG. 5A.

FIG. 5E shows a lengthwise view of the assembled suction device of FIG. 5A.

FIG. 6A shows a side view of a suction device according to an embodiment of the present invention in a retracted state.

FIG. 6B shows a side view of a suction device according to an embodiment of the present invention in an extended state.

### DETAILED DESCRIPTION

The present invention relates to novel suction devices and methods for using the suction device during surgical procedures. In one aspect, the suction device includes an anti-clog mechanism, e.g., an ejection wire, that can be retracted and extended conveniently using a finger of the same hand that is used to hold the suction device. Preferably, the device includes features that allow for convenient ejection of debris from the suction tube using the ejection wire which has knob that controls the ejection wire. The single-hand use of the suction device and ejection wire has particular utility in orthopedic applications, but also has application in surgery generally to free up an additional hand of the user.

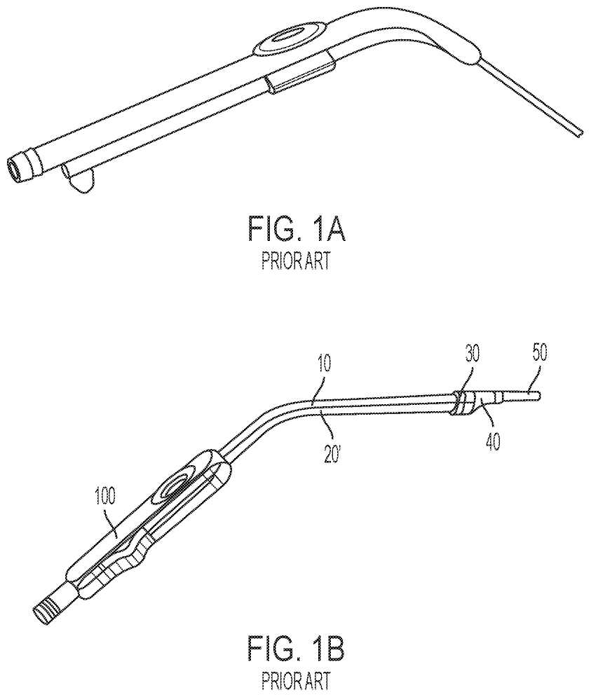

In order to provide a retraction mechanism that can be operated by a finger of the same hand that holds the device, the present inventor has found it desirable to provide a retraction mechanism for the ejection wire that is capable of extending and retracting over a shorter length of travel than existing ejection wires. For example, the Chicago Tip® device as shown in FIG. 1A includes an ejection wire with a length of travel on the order of 8 cm or more. The knob for controlling the ejection wire is provided on the underside of the suction tube when it is held in a horizontal position. In other words, the knob is positioned on lower side of the device or on the side in the direction in which the suction tube bends. In another embodiment, similar suction tubes have been provided with an ejection wire knob on the side of the device as shown in FIG. 1B. Such devices include suction tubes having an ejection wire with a length of travel greater than 25% of the entire length of the device, including >50% of the length of the device. Such devices required a free hand (one not holding the suction tube device) to extend and retract the ejection wire.

The present invention includes suction devices having an ejection wire with a length of travel that is significantly less than what is provided in the prior art. For example, the length of travel of the ejection wire (and ejection wire knob) for the present invention may be on the order of 4 cm or less. For example, the length of travel may range from 1 to 4 cm, or preferably 2 to 3 cm. The length of travel of the knob for controlling the ejection wire may have a range of travel that is 10% or less of the length of the suction tube device. The shorter length of travel provided by the present invention enables operation of the ejection wire knob with one of the user's fingers, and thereby enables operation with a single hand.

The position of the retraction knob in the present invention of being preferably on top or in an upward position when the device is held horizontally further enables extension or retraction with the user's finger of the hand holding the device. Where a suction device is provided with a bend in a first direction, the knob is placed on the side of the suction tube that is opposite the direction of the bend. Prior ejection wire assemblies for suction tubes placed the knob on the side or on the lower side of the device, facing sideways or downward during normal use of the device. This placement in the prior art was chosen so that the retraction knob could be operated by the user's other hand without interfering with the grip the user had on the device by the hand holding the handle.

The present invention also contemplates anti-clog mechanisms that are favorable for preventing bone fragments or chunks of harder material from translating throughout the suction device and suction hose, and that enable a shorter extension/retraction travel than has been found to exist. For example, the suction device may include a “choke point” within the tip of the suction device that is designed to prevent hard materials from entering further into the suction device, where their removal may prove difficult or impossible. The invention further contemplates ejection wire mechanisms that provide an entry point for the ejection wire the is designed to work with the “choke point” such that the device may be used to efficiently remove material clogging the device, or that could pose a threat of clogs after continual use. One aspect of the choke point feature is that it may increase the efficiency with which an ejection wire having a shorter length of travel during operation may be

In another aspect of the invention, the knob for controlling the ejection wire may be placed between the handle and the suction opening, adjacent or near the handle. This placement is necessary to permit operation of the ejection wire with a finger of the same hand that is used to hold the suction device. The knob may also include features, such as gripping mechanism and/or elevated portions that facilitate use of the knob with one finger while the suction tube is being held in the hand at the handle portion. This particular arrangement may further facilitate, for example, a surgeon to remove clogs using the ejection wire using the same hand that holds the device during use, which can potentially free up the other hand for different activities. The present inventor has found that this arrangement is particularly useful in orthopedic surgery applications.

The present invention also includes methods of using the suction devices described herein during surgical procedures. These methods may include (a) removing at least biological fluids and bone fragments from the surgical site using the suction apparatus, and (b) ejecting one or more bone fragments collected in the first portion of the tubular suction channel using the ejection wire. For example, the additional biological fluids may be removed from the surgical site after (b) ejecting one or more bone fragment collected in the first portion of the tubular suction channel.

The materials of the suction device according to embodiments of the invention are preferably plastic material. The ejection wire may be made from a metallic or plastic material. In one aspect, the suction device is generally made from a plastic material which is translucent to X-rays, whereas the ejection wire is made from a radio-opaque material or at least a portion of the ejection wire, e.g., the tip portion, is made from a radio-opaque material. This aspect allows for the device to be seen when using X-ray fluoroscopy or similar imaging techniques.

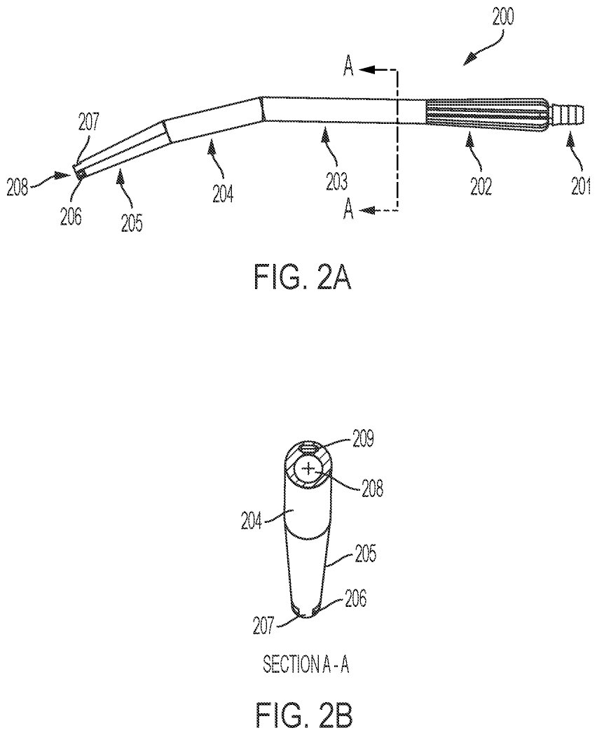

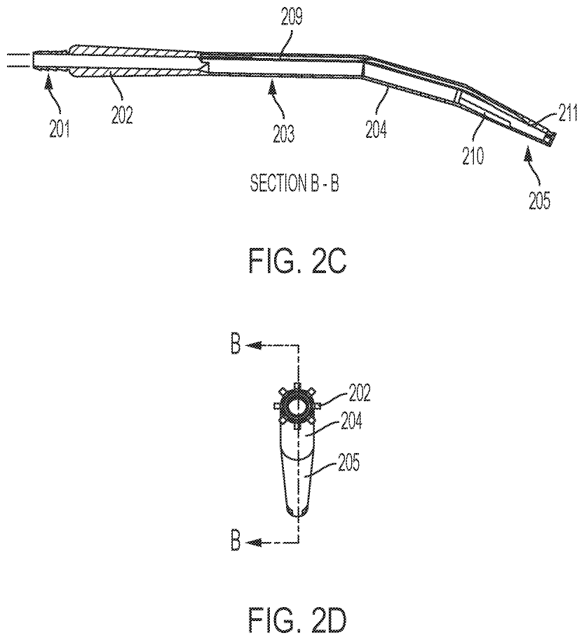

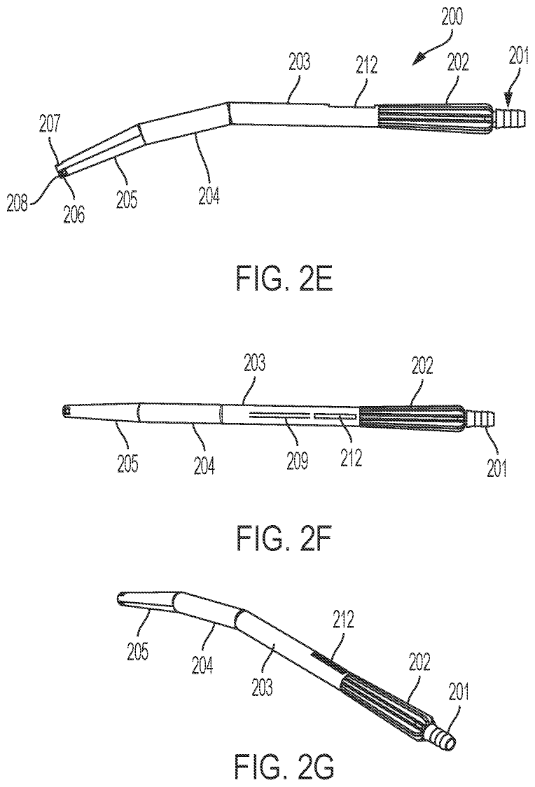

FIG. 2A shows a side view of a suction device 200 according to an embodiment of the invention. The suction device 200 includes a suction hose attachment portion 201 situated at the proximal end of the suction device 200, and a suction opening 208 at the distal end of the suction device 200. A handle portion 202 is located between the attachment portion 201 and suction opening 208, the handle portion 202 being proximate the hose attachment portion 201. The suction device may include distinct sections having a particular angular orientation relative to the longitudinal axis of the suction device. For example, the suction device may include a first section 203, a second section 204, and a third section 205. The suction device may also include holes 206 and projections 207 proximate the suction opening 208 that are designed to prevent airflow blockage in the suction device. In a particular embodiment the projections 207 support a ring structure that defines holes 206 in the side portions of the suction tube.

FIG. 2B shows a cross section of the suction device shown in FIG. 2A along the A-A axis in the first section 203. The cross-section reveals a suction opening 208 within the suction tube, and an ejection wire channel 209 that is incorporated into the wall of the suction tube. The ejection wire channel is designed to allow an injection wire (not shown) to be translated back and forth within the channel. When an operator causes the ejection wire to be advanced, the ejection wire pushes any material clogging the tip portion in the third section 205 to be ejected from the end of the suction device, allowing the device to continued to be used during a surgical procedure.

FIG. 2C shows a cut-out side-view of a suction device 200 according to an embodiment of the invention. This view shows the ejection wire channel 209, which extends from a location proximate the handle portion 202 to an ejection wire entry point 211 within the third portion 205 of the suction device 200. In this embodiment an elevated section 210 is provided within the suction channel 208. The elevated section 210 is distal to the suction opening from the ejection wire entry point 211. In this particular embodiment, the ejection wire entry point is provided between the elevated section 210 and the suction tip opening. The exact location of the ejection wire entry point may be adapted based on the expected use case. In some embodiments, the ejection wire is position just above the edge of the elevated portion 210. In other embodiments, the ejection wire entry point is provided further away from the suction opening such that it is positioned above the middle portion of the elevated section 210. In FIG. 2D, a lengthwise view shows the handle 202 projections extending away from the suction tube, as well as the sections 204 and 205 oriented at various angles relative to the handle portion 202.

FIG. 2E shows a side view of the suction device according to an embodiment of the invention showing the slot 212 where the knob (not shown) is installed. The top-down view of FIG. 2F shows the slot 212 for the knob is aligned with the ejection wire channel 209. FIG. 2G is a perspective view of the device shown in FIG. 2E.

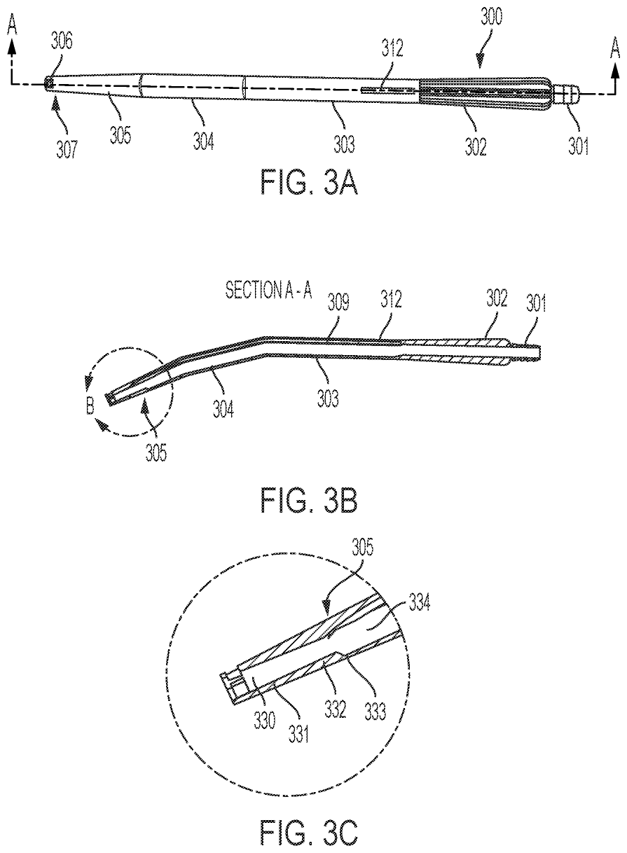

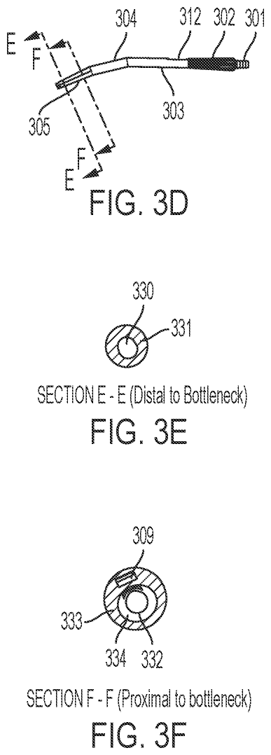

FIG. 3A shows a top-down view of a suction device 300 according to an alternative embodiment that uses a gradual transition to provide a restriction in the suction tube near the ejection wire entry point as shown in FIGS. 3B-3F. In this case, the suction opening 330 in wall 331 of section 305 proximate the distal end of the suction device has a larger diameter than provided at point 332, which can be described as a “choke point” that prevents larger chunks of brittle material from traversing further into the suction device, and potentially into the suction hose connected to the suction device. After point 332, the diameter of the tube widens at point 333 to a suction opening 334 having a greater diameter than provided at 332. This widening can be abrupt or gradual. The entry point of the ejection wire channel should be positioned between the choke point and the suction device opening.

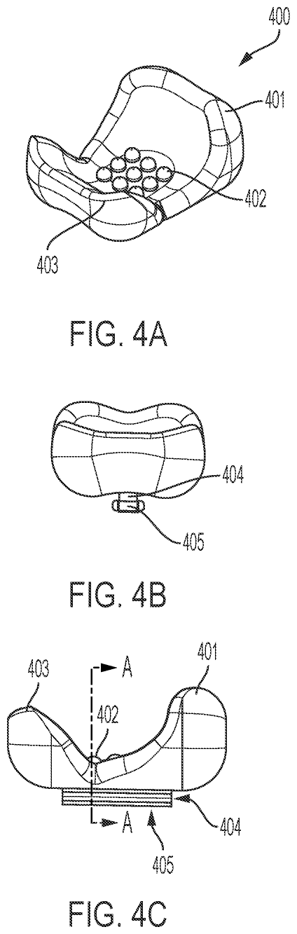

FIG. 4A shows a perspective view of a knob 400 according to an embodiment of the invention. The knob may include an elevated front end 401, gripping features 402, e.g., bumps, and a back end 403. As shown in FIGS. 4B-4C the knob may include a base portion 405 that is designed to engage with the slot in the suction device and a narrower portion that allows the knob to translate back and forth along the slot, while the base portion 405 prevents the knob 400 from detaching from the suction device during use. The base portion is designed to engage with an ejection wire that is provided within the ejection wire channel of the suction device. As noted above, the features of the knob 400 shown in FIGS. 4A-4C can enable the use of the suction device with a single hand, where one finger can be used to translate the knob while the overall device is gripped at the handle by the user of the device (e.g., surgeon).

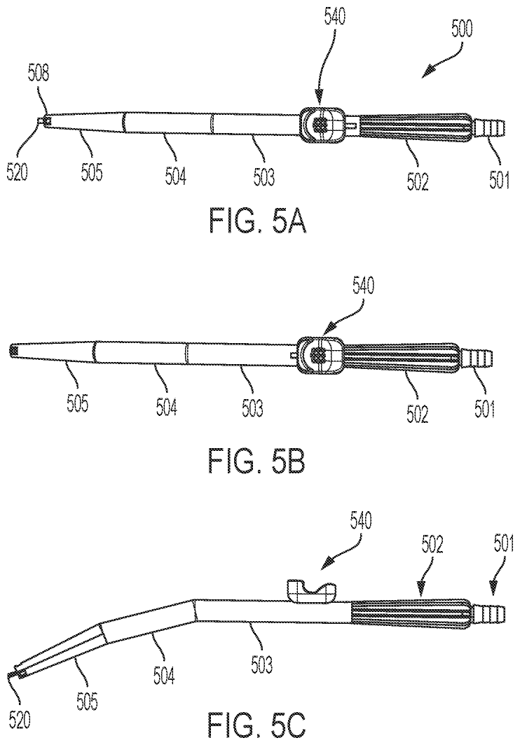

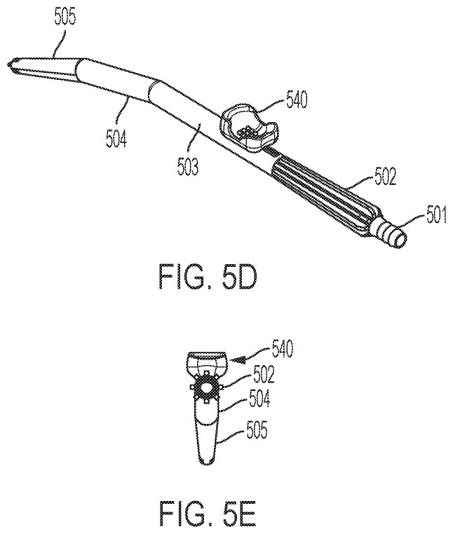

FIG. 5A shows a view of an assembled suction device 500 including a knob 540. The suction device 500 includes a suction hose attachment portion 501 situated at the proximal end of the suction device 500, and a suction opening 508 at the distal end of the suction device 500. A handle portion 502 is located between the attachment portion 501 and suction opening 508, the handle portion 502 being proximate the hose attachment portion 501. The knob 540 is provided proximate the handle portion 502 and shown in FIG. 5A in an extended state such that the ejection wire 520 projects out of the suction opening. FIG. 5B shows the same device with the knob 540 in a retracted position, whereby the ejection wire is no longer projecting from the suction opening. FIG. 5C shows a side view of suction device 500 with the knob in an extended state. FIGS. 5D and 5E show perspective and lengthwise views of the suction device 500.

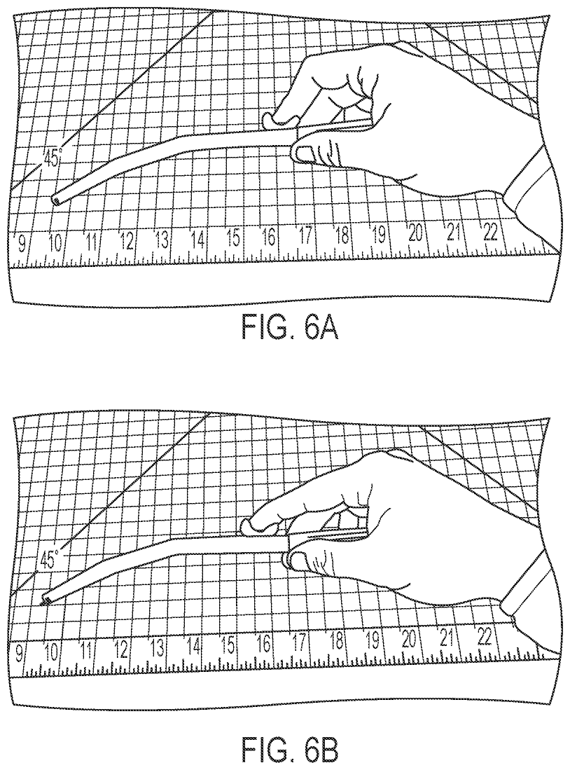

FIG. 6A shows a suction device according to an embodiment of the invention in a retracted state, whereas FIG. 6B shows the device in an extended state. It should be noted that the length of travel shown for this device is on the order of 0.5″ or 1.25 cm. The device can be comfortably held in the hand of the user as shown while the ejection wire knob may be retracted and extended with the index finger of the user's hand that is holding the suction device.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A suction apparatus for the removal of debris from a surgical site, the suction apparatus comprising:
(i) a suction tube having a suction opening at a distal end of the suction apparatus and having an interior defining a tubular suction channel for the removal of debris through suction at the suction opening, the suction tube bending toward a first direction;
(ii) a handle portion adjacent the proximal end of the suction apparatus and connected to the suction tube;
(iii) an ejection wire channel extending from a longitudinal opening in the suction apparatus to a wire exit opening that opens into the tubular suction channel of the suction tube; and
(iv) a knob for controlling an ejection wire within the ejection wire channel, the knob being positioned on a side of the suction tube that is opposite the first direction in which the suction tube is bending.

2. The suction apparatus of claim 1, wherein the knob is positioned proximate the handle portion between the handle portion and the suction tube opening.

3. The suction apparatus of claim 1, wherein the knob has a range of travel that is 4 cm or less.

4. The suction apparatus of claim 1, wherein the knob has a range of travel that is 1 cm to 4 cm.

5. The suction apparatus of claim 1, wherein the tubular suction channel includes a flow restriction.

6. The suction apparatus of claim 5, wherein the wire exit opening into the tubular suction channel is at the location of the flow restriction or is located between the flow restriction and the suction opening at a distal end of the suction apparatus.

7. The suction apparatus of claim 5, wherein the flow restriction is a gradual flow restriction.

8. The suction apparatus of claim 5, wherein the flow restriction is step flow restriction.

9. The suction apparatus of claim 1, wherein the suction apparatus includes an offset ring structure at the suction opening.

10. A suction apparatus for the removal of debris from a surgical site, the suction apparatus comprising:
(i) a suction tube having a suction opening at a distal end of the suction apparatus and having an interior defining a tubular suction channel for the removal of debris through suction at the suction opening;
(ii) a handle portion adjacent the proximal end of the suction apparatus and connected to the suction tube;
(iii) an ejection wire channel extending from a longitudinal opening in the suction apparatus to a wire exit opening that opens into the tubular suction channel of the suction tube; and
(iv) a knob for controlling an ejection wire within the ejection wire channel, wherein the knob has a range of travel that is 4 cm or less.

11. The suction apparatus of claim 10, wherein the knob is positioned proximate the handle portion between the handle portion and the suction tube opening.

12. The suction apparatus of claim 10, wherein the knob has a range of travel that is 1 cm to 4 cm.

13. The suction apparatus of claim 10, wherein the suction tube comprises a bend toward a first direction.

14. The suction apparatus of claim 10, wherein the tubular suction channel includes a flow restriction.

15. The suction apparatus of claim 14, wherein the wire exit opening into the tubular suction channel is at the location of the flow restriction or is located between the flow restriction and the suction opening at a distal end of the suction apparatus.

16. The suction apparatus of claim 10, wherein the suction apparatus includes an offset ring structure at the suction opening.

17. A method cleaning a surgical site with a suction apparatus comprising:
(a) removing at least biological fluids and bone fragments from the surgical site using the suction apparatus, the suction apparatus comprising:
(i) a suction tube having a suction opening at a distal end of the suction apparatus and having an interior defining a tubular suction channel for the removal of debris through suction at the suction opening, the suction tube bending toward a first direction;
(ii) a handle portion adjacent the proximal end of the suction apparatus and connected to the suction tube;
(iii) an ejection wire channel extending from a longitudinal opening in the suction apparatus to a wire exit opening that opens into the tubular suction channel of the suction tube; and
(iv) a knob for controlling an ejection wire within the ejection wire channel, the knob being positioned on a side of the suction tube that is opposite the first direction in which the suction tube is bending; and
(b) ejecting one or more bone fragments collected in the first portion of the tubular suction channel using the ejection wire.

18. The method of claim 17, wherein the additional biological fluids are removed from the surgical site after (b) ejecting one or more bone fragment collected in the first portion of the tubular suction channel.

19. A method cleaning a surgical site with a suction apparatus comprising:
(a) removing at least biological fluids and bone fragments from the surgical site using the suction apparatus, the suction apparatus comprising:
(i) a suction tube having a suction opening at a distal end of the suction apparatus and having an interior defining a tubular suction channel for the removal of debris through suction at the suction opening;
(ii) a handle portion adjacent the proximal end of the suction apparatus and connected to the suction tube;
(iii) an ejection wire channel extending from a longitudinal opening in the suction apparatus to a wire exit opening that opens into the tubular suction channel of the suction tube; and
(iv) a knob for controlling an ejection wire within the ejection wire channel, wherein the knob has a range of travel that is 4 cm or less; and
(b) ejecting one or more bone fragments from the suction device using the ejection wire.

20. The method of claim 19, wherein the additional biological fluids are removed from the surgical site after (b) ejecting one more bone fragment collected in the first portion of the tubular suction channel.
